(19) United States
(12) Patent Application Publication
(10) Pub. No.: US 2024/0386301 A1
(43) Pub. Date: Nov. 21, 2024

(54) WAVEFORM GENERATION IDENTIFYING METHOD AND COMPUTER-READABLE MEDIUM

(71) Applicants: Miyako ASAI, Oosaka (JP); Ryoji HIRANO, Tokyo (JP); Masayuki HIRATA, Oosaka (JP)

(72) Inventors: Miyako ASAI, Oosaka (JP); Ryoji HIRANO, Tokyo (JP); Masayuki HIRATA, Oosaka (JP)

(21) Appl. No.: 18/663,526

(22) Filed: May 14, 2024

(30) Foreign Application Priority Data
May 19, 2023 (JP) 2023-083507

(51) Int. Cl. G06N 7/01; A61B 5/00

(52) U.S. Cl. CPC G06N 7/01; A61B 5/4094; A61B 5/7264; A61B 5/7275

(57) ABSTRACT

A waveform generation identifying method including: acquiring waveform data of biological signals measured by a plurality of sensors; calculating probability information of appearance of IEDs (interictal epileptiform discharges) from a deep learning model trained using the waveform data with labels indicating whether the characteristic waveform information appears or not; and first extracting a time and a sensor at which the characteristic waveform information appears in the waveform data, based on the probability information. The calculating includes: second extracting a feature map indicating waveform data characteristics from the waveform data; generating an attention map indicating an important region in IED recognition, from the feature map; and identifying the probability information by inputting information obtained by multiplying the feature map by the attention map.

### CROSS-REFERENCE TO RELATED APPLICATION

The present application claims priority under 35 U.S.C. § 119 to Japanese Patent Application No. 2023-083507, filed on May 19, 2023. The contents of which are incorporated herein by reference in their entirety.

### BACKGROUND OF THE INVENTION

### 1. Field of the Invention

The present invention relates to a waveform generation identifying method and a computer-readable medium.

### 2. Description of the Related Art

In clinical diagnosis of epilepsy using a magneto-encephalography or an electro-encephalography, the localization of brain epileptic lesions is evaluated with a technique called an equivalent current dipole method. The estimation of current sources (dipole) generating the magnetic field measured on the scalp is carried out by the equivalent current dipole method. In order to estimate the dipole, it is imperative to narrow down, from time series data detected by a plurality of sensors, the time when characteristic waveform information called interictal epileptiform discharge (IED) is generated and the sensor where the waveform information appears. In the analysis of epilepsy with magnetoencephalography and electroencephalography in the related art, a physician or technician manually conducts IED searches. However, the manual analysis of individual IEDs is difficult and time-consuming because the detection is conducted for 30 minutes per person at a sampling frequency of about 2,000 (Hz) with a large number of sensors. Therefore, a method for identifying a time and sensors at which IED is generated with deep learning is proposed.

As a technique for extracting (identifying) a time and a sensor at which the characteristic waveform information (hereinafter, simply referred to as “characteristic waveform information”) of such IED is generated, a technique of calculating a probability at which the characteristic waveform information appears for each time of waveform data detected by each sensor using a deep learning algorithm, such as Object Detection or Segmentation, and identifying the time and the sensor at which the waveform information appears has been disclosed (for example, Japanese Unexamined Patent Application Publication No. 2021-069929).

However, a conventional technique using deep learning to extract the time and sensor at which characteristic waveform information on IED appears may present the results of the extracted time and sensor, which are not matched with the results of analysis conducted by experts such as doctors, thereby causing issues that may lead to false detection and missed detection.

### SUMMARY OF THE INVENTION

According to an aspect of the present invention, a waveform generation identifying method including: acquiring waveform data of biological signals measured by a plurality of sensors; calculating probability information of appearance of IEDs (interictal epileptiform discharges) from a deep learning model trained using the waveform data with labels indicating whether characteristic waveform information appears or not; and first extracting a time and a sensor at which the characteristic waveform information appears in the waveform data, based on the probability information. The calculating includes: second extracting a feature map indicating waveform data characteristics from the waveform data; generating an attention map indicating an important region in IED recognition, from the feature map; and identifying the probability information by inputting information obtained by multiplying the feature map by the attention map.

### BRIEF DESCRIPTION OF THE DRAWINGS

FIG. 1 is a diagram illustrating an example of the overall configuration of a biological signal measurement system according to a first embodiment;

FIG. 2 is a diagram illustrating an example of the configuration of functional blocks of a server according to the first embodiment;

FIG. 3 is a diagram illustrating an example of the hardware configuration of an information processing apparatus according to the first embodiment;

FIG. 4 is a diagram illustrating an example of the configuration of functional blocks of the information processing apparatus according to the first embodiment;

FIG. 5 is a diagram illustrating an example of the model configuration of a probability calculation unit of the information processing apparatus according to the first embodiment;

FIG. 6 is a diagram illustrating an example of an IED probability map;

FIG. 7 is a diagram illustrating an example of the IED probability map after threshold processing;

FIG. 8 is a diagram illustrating an example of the IED probability map after extraction processing using an attention map;

FIGS. 9A to 9C are conceptual diagrams illustrating an example of a method for expanding the number of extracted sensors;

FIG. 10 is a flowchart illustrating an example of the flow of training processing executed by the information processing apparatus according to the first embodiment;

FIG. 11 is a flowchart illustrating an example of the flow of dipole estimation processing executed by the information processing apparatus according to the first embodiment;

FIG. 12 is a diagram illustrating an example of the model configuration of a probability calculation unit of the information processing apparatus according to a second embodiment;

FIG. 13 is a diagram illustrating an example of the model configuration of a probability map calculation unit of the information processing apparatus according to a third embodiment; and

FIG. 14 is a diagram illustrating an example of the model configuration of a probability map calculation unit of the information processing apparatus according to a fourth embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to provide a waveform generation identifying method and a computer-readable medium which enables the extraction of a more accurate analysis result for a time and a sensor at which characteristic waveform information of IED appears.

Hereinbelow, embodiments of a method for identifying waveform generation and a computer program according to the present invention will be described in details with reference to the drawings. The present invention is not limited by the following embodiments, and components in the following embodiments include those readily conceivable by a person skilled in the art, those substantially identical to the components, and those in the so-called equivalent range. Furthermore, various omissions, substitutions, modifications, and combinations of the components may be made without departing from the gist of the following embodiments.

### First Embodiment

### Overall Configuration of Bio-Signal Measurement System

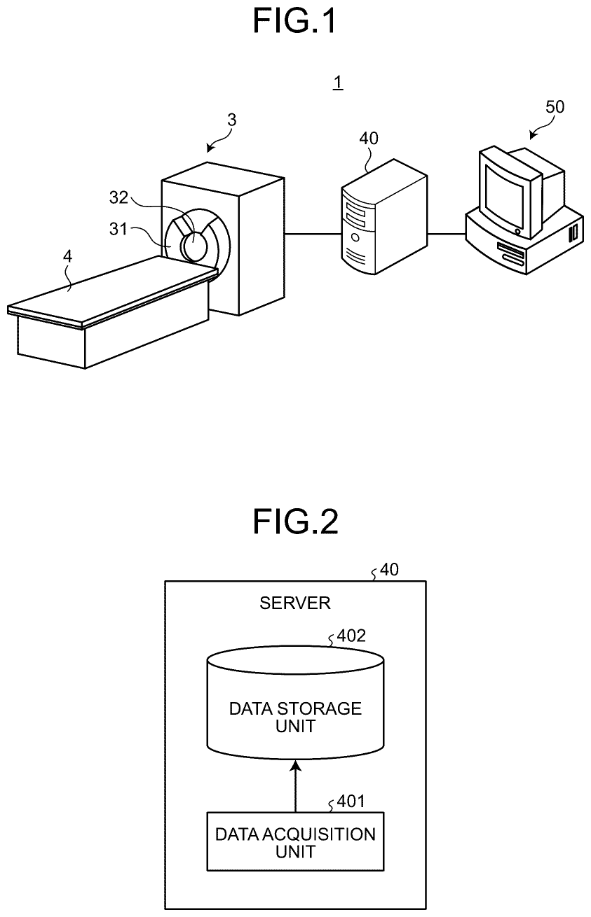

FIG. 1 is a diagram illustrating an example of the overall configuration of a biological signal measurement system according to a first embodiment. FIG. 2 is a diagram illustrating an example of the configuration of functional blocks of a server according to the first embodiment. The overall configuration of a biological signal measurement system 1 according to the present embodiment will be described with reference to FIGS. 1 and 2.

The biological signal measurement system 1 measures various types of biological signals (for example, magneto-encephalography (MEG) signals and electro-encephalography (EEG) signals) of a subject being tested from a specific source (biological site) and displays the measured signals. The biological signals to be measured are not limited to magneto-encephalography signals or electro-encephalography signals, and may also be electrical signals generated in response to cardiac activity (electrical signals that can be expressed as an electrocardiogram) and other signals, for example.

As illustrated in FIG. 1, the biological signal measurement system 1 includes a measurement device 3 that measures one or more biological signals from a subject being tested, a server 40 that records one or more biological signals measured by the measurement device 3, and an information processing apparatus 50 that functions as a biological signal display device and analyzes the one or more biological signals recorded in the server 40. The measurement device 3 is, for example, a magneto-encephalography that measures the brain magnetic fields or magneto-encephalography signals (an example of biological signals) generated at the stimulation timing. Although the server 40 and the information processing apparatus 50 are separately illustrated in FIG. 1, for example, at least some of the functions of the server 40 may be incorporated into the information processing apparatus 50.

In the example in FIG. 1, a subject being tested (subject being measured) lies down in a supine position on a measuring table 4 with electrodes (or sensors) for electro-encephalography measurement attached to the subject's head region, and then puts the head region in a recess 32 of a dewar 31 of the measurement device 3 The dewar 31 is a cryogenic container with liquid helium. The inside of the recess 32 of the dewar 31 is provided with a large number of magnetic sensors (for example, superconducting quantum interference device (SQUID) sensors) for magneto-encephalography. The measurement device 3 collects electro-encephalography signals from the electrodes and magneto-encephalography signals from the magnetic sensors and outputs time series waveform data of the collected electro-encephalography signals and magneto-encephalography signals (which may be hereinafter simply referred to as waveform data) to the server 40. The waveform data output to the server 40 is read, displayed, and analyzed by the information processing apparatus 50. While the dewar 31 into which the magnetic sensors are incorporated and the measuring table 4 are usually disposed in a magnetic shielding room, the magnetic shielding room is not illustrated in FIG. 1 for the convenience of explanation.

The information processing apparatus 50 analyzes the waveform data of magneto-encephalography signals from the magnetic sensors and the waveform data of electro-encephalography signals from a plurality of the electrodes. For example, the information processing apparatus 50 displays the waveform data of the magneto-encephalography signals and the waveform data of the electro-encephalography signals in synchronization on the same time axis. The electro-encephalography signal represents electrical activity of a nerve cell (the flow of an ionic charge generated at a dendrite of a neuron during synaptic transmission) as a voltage value between the electrodes. The magneto-encephalography signal represents minute magnetic field fluctuations generated by electrical activity of a brain.

The server 40 includes a data acquisition unit 401 and a data storage unit 402, as illustrated in FIG. 2.

The data acquisition unit 401 is a functional unit that periodically acquires waveform data such as magneto-encephalography signals and EEG signals measured by the measurement device 3. The waveform data includes time series data of respective magneto-encephalography signals measured by the magnetic sensors in the dewar 31 of the measurement device 3 and respective time series data measured by the electrodes for electro-encephalography measurement attached to the head region of the subject being tested (subject being measured).

The data storage unit 402 is a functional unit that stores waveform data acquired from the measurement device 3.

Although FIG. 1 illustrates the configuration in which the measurement device 3 is directly connected to the server 40, and the server 40 is directly connected to the information processing apparatus 50, the configuration in which these components can communicate data with each other via a network may be employed. The network connection method can be either wired or wireless. The server 40 may also be on a network, for example, and may be configured to be cloud-connected.

### Hardware Configuration of Information Processing Apparatus

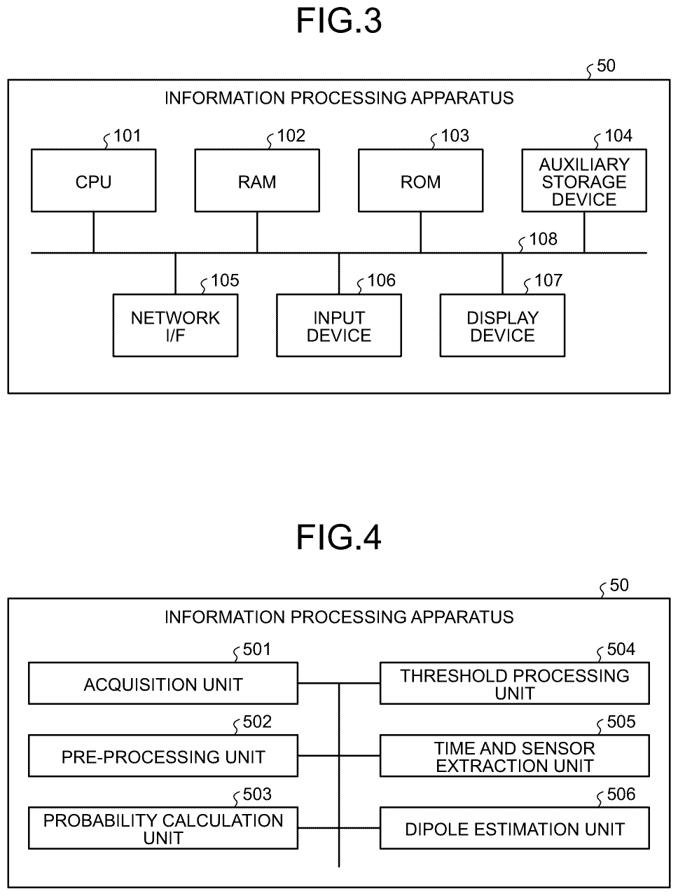

FIG. 3 is a diagram illustrating an example of the hardware configuration of the information processing apparatus according to the first embodiment. With reference to FIG. 3, the hardware configuration of the information processing apparatus 50 according to the present embodiment will be described.

As illustrated in FIG. 3, the information processing apparatus 50 provided with a central processing unit (CPU) 101, a random access memory (RAM) 102, a read only memory (ROM) 103, an auxiliary storage device 104, a network I/F 105, an input device 106, and a display device 107.

The CPU 101 is an arithmetic unit that controls the overall operations of the information processing apparatus 50. The RAM 102 is a volatile memory used as the work area for the CPU 101. The ROM 103 is a nonvolatile memory that stores computer programs for the information processing apparatus 50.

The auxiliary storage device 104 is a storage device such as a hard disk drive (HDD) or solid state drive (SSD) that stores various data, computer programs, and the like.

The network I/F 105 is an interface for data communication via a network with external devices such as the server 40. The network I/F 105 is, for example, a network interface card (NIC) or the like, which is compatible with Ethernet (registered trademark) and capable of wired or wireless communication in accordance with Transmission Control Protocol (TCP)/Internet Protocol (IP) or the like.

The input device 106 is input function of a touch panel, mouse, keyboard, and the like for selecting letters, numbers, and various instructions, and for moving the cursor.

The display device 107 is a display made with liquid crystal, organic electro-luminescent (EL), or the like, which display various information such as cursors, menus, windows, text, or images.

The CPU 101, RAM 102, ROM 103, auxiliary storage device 104, network I/F 105, input device 106, and display device 107 described above are communicably connected to each other via a bus 108 such as address bus or data bus.

The hardware configuration of the information processing apparatus 50 illustrated in FIG. 3 is an example and is not necessary to include all of the components illustrated in FIG. 3, or may include other components.

### Configuration of Functional Blocks and Operation of Information Processing Apparatus

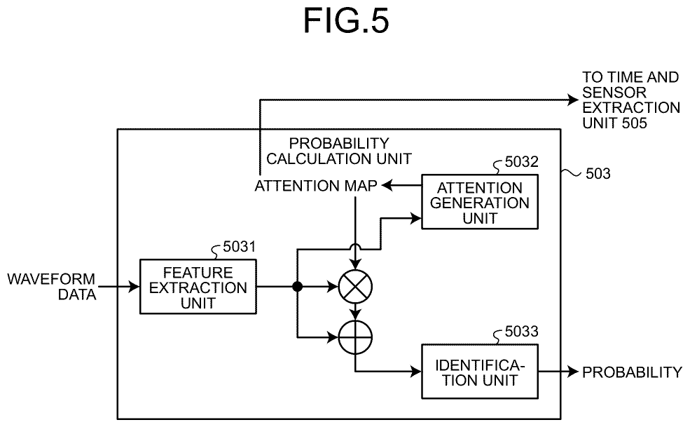

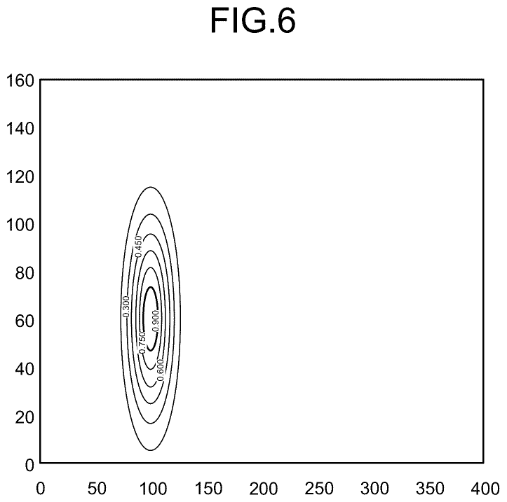

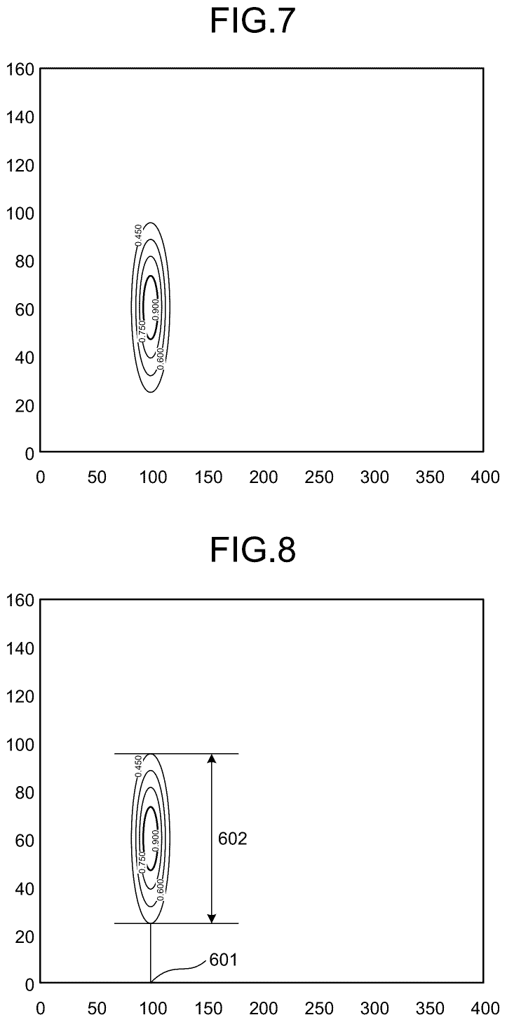

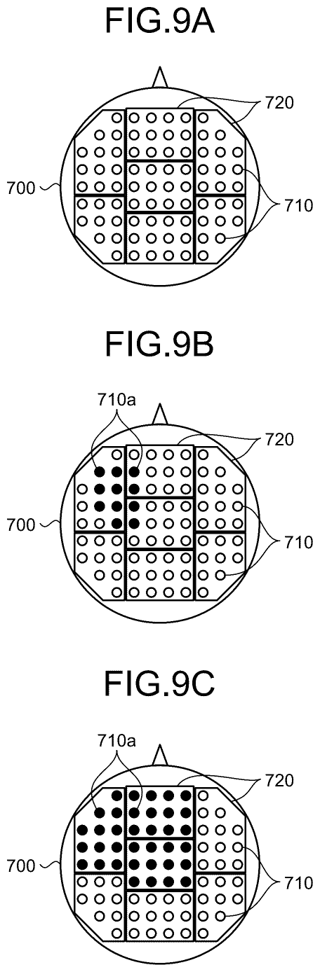

FIG. 4 is a diagram illustrating an example of the configuration of functional blocks of the information processing apparatus according to the first embodiment. FIG. 5 is a diagram illustrating an example of the model configuration of a probability calculation unit of the information processing apparatus according to the first embodiment. FIG. 6 is a diagram illustrating an example of an IED probability map. FIG. 7 is a diagram illustrating an example of the IED probability map after threshold processing. FIG. 8 is a diagram illustrating an example of the IED probability map after extraction processing using an attention map. FIGS. 9A to 9C are conceptual diagrams illustrating an example of a method for expanding the number of extracted sensors. With reference to FIGS. 4 to 9, the configuration of the functional blocks and the operation of the information processing apparatus 50 according to the present embodiment will be described. Specifically, in FIG. 5, the details of the model configuration of a probability calculation unit 503 based on an attention branch network (ABN) (see, for example, Fukui, Hiroshi, Tsubasa Hirakawa, Takayoshi Yamashita, and Hironobu Fujiyoshi. 2019. “Attention Branch Network: Learning of Attention Mechanism for Visual Explanation.” In Proceedings of the IEEE/CVF Conference on Computer Vision and Pattern Recognition, 10705-14.), which is an example of a deep learning algorithm, will be described.

As illustrated in FIG. 4, the information processing apparatus 50 includes an acquisition unit 501, a pre-processing unit 502, the probability calculation unit 503, a threshold processing unit 504, a time and sensor extraction unit 505, and a dipole estimation unit 506.

The acquisition unit 501 is a functional unit that acquires waveform data of magneto-encephalography signals and electro-encephalography signals from the server 40 via the network I/F 105.

The pre-processing unit 502 is a functional unit that executes, on the waveform data acquired by the acquisition unit 501, pre-processing such as extraction and expansion of sensors, the application of downsampling and a frequency filter, artifact removal, defective channel processing, time window (predetermined interval) clipping, and standardization of magnetic field data.

The extraction and expansion of sensors are calculated by using all the sensors or only a certain number of sensor groups that have been grouped in advance when the probability value of IED is calculated, as described below. As for the grouping of sensors, a grouping of sensors may be set in line with the anatomical standards such as the temporal lobe and the frontal lobe, or any number of adjacent sensors may be simply grouped. In addition, in a case in which the number of sensors used in the calculation is smaller than the number of sensors used in a training process described later, the number of imaginary sensors can be expanded.

The downsampling is applied with the aim of matching the sampling frequency used during the training. Regarding the frequency filter, the same frequency filter used for filtering applied during the same training is applied. Examples of commonly used filters include a 35 (Hz) low-pass filter, a 3 (Hz) to 35 (Hz) band-pass filter, and other filters.

As for artifact removal, ICA (see, for example, E. Javier, H. Roberto, A. Daniel, F. Alberto, and L. C. Miguel, “Artifact removal in magnetoencephalogram background activity with independent component analysis,” IEEE Trans Biomed Eng, vol. 54, no. 11, pp. 1965-1973, 2007.), DSSP (see, for example, K. Sekihara, Y. Kawabata, S. Ushio, S. Sumiya, S. Kawabata, Y. Adachi, and S. S. Nagarajan, “Dual signal subspace projection (DSSP): a novel algorithm for removing large interference in biomagnetic measurements,” Journal of Neural Engineering, vol. 13, no. 3, p.), or the like is applied with the aim of eliminating the cardiac artifact or the artifact attributed to blinking and body motion, and other artifacts.

The defective channel processing implies excluding magnetic sensors at which magnetic field changes have observed to exceed a preset threshold value or performing interpolation using surrounding sensor values.

As for the time window clipping, there are methods such as a method of shifting only the portions equivalent to the lengths of time windows without any overlapping to clip the time windows; a method of overlapping halves of the lengths of time windows; and a method of overlapping quarters of the lengths of time windows. In the case of overlapping, an arithmetic average of the overlapping portions is carried out during the calculation of probability values of IED described later.

The standardization of magnetic field data is applied such that the average of 0 and the dispersion of 1 are achieved in the clipped time window. In addition to the standardization, a method of normalizing the preset range of the magnetic field to achieve the range from −1 to 1 may be used.

The probability calculation unit 503 is a functional unit that calculates probability values (in other words, values between 0 and 1) (an example of probability information) at which the characteristic waveform information of IED appears in the waveform data that has been pre-processed by the pre-processing unit 502. Specifically, the probability calculation unit 503 performs the inference with a trained model based on ABN, which is an example of a deep learning algorithm, and calculates the probability values. The ABN consists of two parts obtained by dividing a convolutional neural network (CNN) into the first part serving as a feature extractor and the second part serving as a perception branch that outputs probability values based on the feature map weighted by the attention map output from the attention branch. The characteristic waveform information of IED indicates waveform information such as spike waves, spikes and waves, polyspikes and waves, sharp waves, and other waves. Instead of inferring the probability values from the input waveform data, the trained model described above may output a probability map (an example of probability information) as illustrated in FIG. 6 below, which indicates the distribution of the probability at which the characteristic waveform information of IED appears. The probability calculation unit 503 can also construct a probability map according to the correspondence between the time and the sensors, and the probability values by inputting the waveform data with specific sensors, which is clipped at a predetermined time (time window) by the pre-processing unit 502, into the trained model to obtain the probability values as output.

The probability calculation unit 503 includes a feature extraction unit 5031, an attention generation unit 5032, and an identification unit 5033, as illustrated in FIG. 5.

The feature extraction unit 5031 is a functional unit that inputs the waveform data that has been pre-processed by the pre-processing unit 502 and generates a feature map that indicates the distribution of characteristic information extracted by a predetermined kernel for the waveform data. The feature extraction unit 5031 corresponds to the feature extractor in the first part of ABN.

The attention generation unit 5032 is a functional unit that generates an attention map indicating an important region in IED recognition from the feature map generated by the feature extraction unit 5031. The attention generation unit 5032 corresponds to the attention branch of ABN. Here, the feature map and the attention map have the same size. The attention map generated by the attention generation unit 5032 is output to the time and sensor extraction unit 505.

The identification unit 5033 is a functional unit that inputs information by multiplying the feature map output from the feature extraction unit 5031 by the attention map output from the attention generation unit 5032 to obtain the weighted feature map and furthermore adding the feature map itself to the weighted feature map, and identifies (calculates) the probability values at which the characteristic waveform information of IED appears based on the input information. The identification unit 5033 corresponds to the Perception branch of ABN.

As described above, the model trained by ABN of the probability calculation unit 503 is trained using the feature map weighted by multiplying the feature map related to the waveform data by the attention map indicating the important region in IED recognition. Accordingly, the weighted feature map is input to the identification unit 5033, which is the perception branch, to enable the training focusing on the important region in IED recognition indicated in the attention map and the output of the probability value of IED with high accuracy.

Although the information to be input to the identification unit 5033 is obtained by the feature map being weighted by multiplying the feature map by the attention map and furthermore adding the feature map itself thereto, this addition is not necessarily performed. However, the feature map with the region where the attention map responds strongly can be highlighted by adding the feature map itself. There is also the effect of not losing the feature map with the region where a value is 0 in the attention map.

Here, an example of the probability map, assuming that the probability map is calculated by the probability calculation unit 503, is illustrated in FIG. 6. In FIG. 6, the horizontal axis represents a time, and the vertical axis represents the index of a magnetic sensor (or electrode) (which may be hereinafter referred to as the sensor). In the example illustrated in FIG. 6, it is estimated that the IED appears at an index of time of 100 and an index of sensor around 40 to 80. In FIG. 6, although the IEDs are represented in shades of black and white according to the probability of IEDs, the IEDs can also be indicated in color, with the color changing according to the probability of IEDs.

Return to FIG. 4 to continue the explanation.

The threshold processing unit 504 is a functional unit that determines whether the probability is equal to or greater than a predetermined threshold value with respect to the probability value calculated by the probability calculation unit 503. Provided that the probability map is calculated by the probability calculation unit 503, the threshold processing unit 504 can also narrow down whether the probability values are equal to or greater than a predetermined threshold value with respect to the probability map. An example of the probability map with such narrow down performed is illustrated in FIG. 7. In the example illustrated in FIG. 7, the threshold processing unit 504 uses “0.5” as the threshold value. In other words, the probability map illustrated in FIG. 7 corresponds to extracted regions where the characteristic waveform information of IED is present with a probability of 50% or more.

The time and sensor extraction unit 505 is a functional unit that extracts, from the probability values determined as equal to or greater than the threshold value by the threshold processing unit 504, the time and the sensors related to regions strongly responding in the attention map, generated by the attention generation unit 5032 of the probability calculation unit 503, that is, regions to be watched indicated in the attention map. Here, provided that the probability map is calculated by the probability calculation unit 503, an example of the probability map with the regions strongly responding in the attention map used by the time and sensor extraction unit 505 is illustrated in FIG. 8. In the example illustrated in FIG. 8, the time and sensor extraction unit 505 extracts a time 601 and a sensor group 602 by using the probability map with the regions strongly responding in the attention map.

As another method for extracting a sensor by the time and sensor extraction unit 505, a group of a sensor can be defined in advance and all sensors belonging to the group to which the extracted sensor belongs as the extracted sensors can be expanded to increase the stability of the dipole estimation. Here, the group is basically set in light with anatomical standards such as near the temporal lobe or near the frontal lobe, but any number of adjacent regions may be simply grouped in a single group.

The time and sensor extraction unit 505 is not limited to extracting the time and sensors using the attention map with respect to the probability value determined as being equal to or greater than the threshold value by the threshold processing unit 504. The time and sensors may be extracted using the attention map with respect to the probability value calculated by the probability calculation unit 503.

Here, an example of a sensor expansion method is illustrated in FIGS. 9A to 9C. FIG. 9A illustrates a head region 700 of the subject being tested, sensors 710 disposed to cover the head region 700, and predetermined groups 720 of the sensors 710. Each sensor 710a, represented by a black circle in FIG. 9B, represents a sensor extracted at the stage in which narrowing down is performed by the threshold processing unit 504. Although dipole estimation can also be carried out by using only the sensors 710a extracted at the stage in which these sensors are narrowed down, sensor expansion can also be implemented to increase the stability of the dipole estimation solution as described above. FIG. 9C is the result of the expansion of the number of the sensors. In other words, as illustrated in FIG. 9C, the sensors 710a extracted at the stage in which the threshold value is determined by the threshold processing unit 504 are expanded to all the sensors 710 belong to the groups 720 to which the sensors 710a belong. Accordingly, the stability of dipole estimation can be increased.

The dipole estimation unit 506 is a functional unit that performs dipole estimation (see, for example, M. Scherg, “Fundamentals of dipole source potential analysis,” in Auditory Evoked Magnetic Fields and Potentials, M. Hoke, F. Grandori, and G. L. Romani, Eds. Basel, Switzerland: Karger, 1989, vol. 6 s) using waveform data corresponding to the time and sensors extracted by the time and sensor extraction unit 505.

The CPU 101 illustrated in FIG. 3 executes a computer program to implement the above-described acquisition unit 501, pre-processing unit 502, probability calculation unit 503, threshold processing unit 504, time and sensor extraction unit 505, and dipole estimation unit 506. Some or all of the functional units of the acquisition unit 501, pre-processing unit 502, probability calculation unit 503, threshold processing unit 504, time and sensor extraction unit 505, and dipole estimation unit 506 may be implemented not by a computer program, which is software, but by a hardware circuit (integrated circuit) such as a field-programmable gate array (FPGA) or application specific integrated circuit (ASIC).

Each functional unit of the information processing apparatus 50 illustrated in FIG. 4 is a conceptually represented function and is not limited to such a configuration. For example, a plurality of the functional units illustrated as independent functional units in the information processing apparatus 50 illustrated in FIG. 4 may be configured as a single functional unit. On the other hand, the function of a single functional unit in the information processing apparatus 50 illustrated in FIG. 4 may be divided into multiple functions to be configured as a plurality of functional units.

### Flow of Training Processing of Information Processing Apparatus

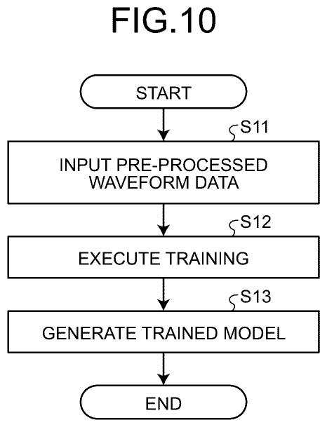

FIG. 10 is a flowchart illustrating an example of the flow of training processing executed by the information processing apparatus according to the first embodiment. With reference to FIG. 10, the flow of the training processing executed by the information processing apparatus 50 according to the present embodiment will be described.

### Step S11

The acquisition unit 501 acquires waveform data that serves as labeled training data for training in the probability calculation unit 503. Next, the pre-processing unit 502 clips out each fixed interval (for example, 1 second) as pre-processing to the waveform data acquired by the acquisition unit 501. The label of IED positive case is added to the pre-processed waveform data for each fixed interval when the characteristic waveform information of IED appears, and the label of IED negative case is added when the characteristic waveform information of IED does not appear, which are used as the labeled training data (training data) for training in the probability calculation unit 503. For example, as the waveform data labeled with IED-positive case, waveform data already used in dipole estimation may be used. The probability calculation unit 503 then inputs the labeled training data. Then, the processing proceeds to step S12.

### Step S12

The probability calculation unit 503 executes the training processing based on the ABN algorithm to cause the identification unit 5033 to output the IED probability values by using the input labeled training data. Then, the processing proceeds to step S13.

### Step S13

The probability calculation unit 503 then generates a trained model of ABN as a result of the training processing at step S12. The generated trained model is used for inference to detect IEDs for unknown waveform data.

### Flow of Dipole Estimation Processing of Information Processing Apparatus

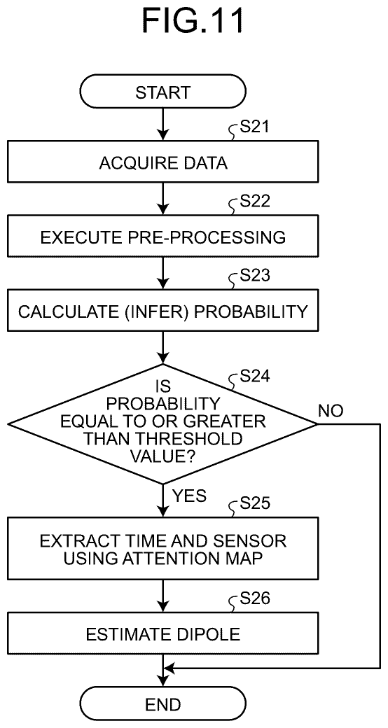

FIG. 11 is a flowchart illustrating an example of the flow of dipole estimation processing executed by the information processing apparatus according to the first embodiment. With reference to FIG. 11, the flow of dipole estimation processing executed by the information processing apparatus 50 according to the present embodiment will be described.

### Step S21

The acquisition unit 501 acquires waveform data of magneto-encephalography signals and electro-encephalography signals from the server 40 via the network I/F 105. Then, the processing proceeds to step S22.

### Step S22

The pre-processing unit 502 executes, on the waveform data acquired by the acquisition unit 501, pre-processing such as extraction and expansion of sensors, the application of downsampling and a frequency filter, artifact removal, defective channel processing, time window clipping, and standardization of magnetic field data. Then, the processing proceeds to step S23.

### Step S23

The probability calculation unit 503 inputs the waveform data that has been pre-processed by the pre-processing unit 502 and calculates (infers) IED probability values with the trained model of ABN generated by the training processing described above. The specific processing of calculating the probability values of IED in the probability calculation unit 503 is as described above in FIG. 5. Then, the processing proceeds to step S24.

### Step S24

The threshold processing unit 504 determines whether the probability values are equal to or greater than the predetermined threshold value with respect to the probability values calculated by the probability calculation unit 503. Provided that the probability values are equal to or greater than the predetermined threshold value (Yes at step S24), the processing proceeds to step S25, and provided that the probability values are smaller than the predetermined threshold value (No at step S24), the dipole estimation processing is terminated as no characteristic waveform information of IED appears in the waveform data input to the probability calculation unit 503.

### Step S25

The time and sensor extraction unit 505 extracts, from the probability values determined as equal to or greater than the threshold value by the threshold processing unit 504, a time and sensors related to regions strongly responding in the attention map, generated by the attention generation unit 5032 of the probability calculation unit 503. Then, the processing proceeds to step S26.

### Step S26

The dipole estimation unit 506 performs dipole estimation using waveform data corresponding to the time and sensors extracted by the time and sensor extraction unit 505. Accordingly, the dipole estimation processing is terminated.

As described above, in the information processing apparatus 50 of the present embodiment, the acquisition unit 501 acquires the waveform data of biological signals measured by a plurality of the sensors; the probability calculation unit 503 calculates a probability value in which characteristic waveform information appears in the waveform data by using the trained model of a deep learning (for example, ABN) trained using, as the labeled training data, the waveform data with a label indicating whether the characteristic waveform information of IED appears in the waveform data; and the time and sensor extraction unit 505 extracts a time and sensors at which the characteristic waveform information appears in the waveform data based on the probability value, in which, in the probability calculation unit 503, the feature extraction unit 5031 extracts a feature map indicating waveform data characteristics from the waveform data, the attention generation unit 5032 generates an attention map indicating an important region in IED recognition from the feature map, and the identification unit 5033 identifies the probability value by inputting information obtained by multiplying the feature map by the attention map. Accordingly, the more accurate analysis results can be extracted with respect to the time and sensors at which the characteristic waveform information of IED appears.

### Second Embodiment

The biological signal measurement system 1 according to a second embodiment will be described, focusing on the differences from the biological signal measurement system 1 according to the first embodiment. In the present embodiment, regarding the waveform data in which characteristic waveform information of IED appears in addition to the labeled waveform data, as the labeled training data, a method of using information (master information) on a time (sampling time) and a sensor recorded during the dipole estimation will be described. The overall configuration of the biological signal measurement system 1 and the hardware configuration of the information processing apparatus 50 according to the present embodiment are the same as those described in the first embodiment.

### Configuration of Functional Blocks and Operation of Information Processing Apparatus

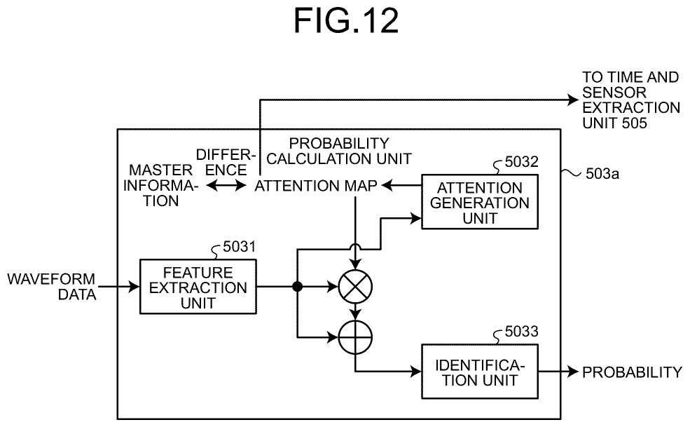

FIG. 12 is a diagram illustrating an example of the model configuration of the probability calculation unit of the information processing apparatus according to the second embodiment. With reference to FIG. 12, the configuration of the functional blocks and the operation of the information processing apparatus 50 according to the present embodiment will be described.

The information processing apparatus 50 according to the present embodiment has a configuration in which the probability calculation unit 503 in the configuration of the functional blocks illustrated in FIG. 4 described above is replaced by a probability calculation unit 503a illustrated in FIG. 12. The probability calculation unit 503a includes a feature extraction unit 5031, an attention generation unit 5032, and an identification unit 5033, as illustrated in FIG. 12. The functions of the feature extraction unit 5031 and the identification unit 5033 are the same as those of the information processing apparatus 50 according to the first embodiment described above.

The attention generation unit 5032 is a functional unit that generates an attention map indicating an important region in IED recognition from the feature map generated by the feature extraction unit 5031. Here, the feature map and the attention map have the same size. The attention map generated by the attention generation unit 5032 is output to the time and sensor extraction unit 505.

Here, regarding the waveform data in which characteristic waveform information of IED appears in addition to the labeled waveform data, as the labeled training data, the model trained by ABN of the probability calculation unit 503a uses information on a time (sampling time) and a sensor recorded during the dipole estimation, which is prepared in advance. In this case, the information on the time (sampling time) and the sensor is referred to as the master information. The probability calculation unit 503a then compares the attention map generated by the attention generation unit 5032 to the master information and performs training so that the difference between the attention map and the master information is reduced to generate a trained model.

In the detection of IED using data from epileptic patients, the result of analysis by an expert such as a neurophysician or medical technologist is usually used as the master information serving as the labeled training data. In this way, since the training is performed with the master information that reflects the knowledge of experts included in the labeled training data, the experts' knowledge can be embedded into the trained model. Accordingly, the more accurate analysis results can be extracted with respect to the time and sensors at which the characteristic waveform information of IED appears.

As the initial weight of the trained model, the weights of the attention generation unit 5032 (attention branch) and the identification unit 5033 (perception branch) in FIG. 12 are fine-tuned (see, for example, Mitsuhara, Masahiro, Hiroshi Fukui, Yusuke Sakashita, Takanori Ogata, Tsubasa Hirakawa, Takayoshi Yamashita, and Hironobu Fujiyoshi. 2019. “Embedding Human Knowledge into Deep Neural Network via Attention Map.” arXiv (cs.CV). arXiv.), to enable the training with further higher accuracy. The fine-tuning may also be performed by manually modifying the important region in IED recognition after the confirmation of the attention map output from the attention generation unit 5032 and using the modified attention map instead of using the master information prepared in advance.

### Third Embodiment

The biological signal measurement system 1 according to a third embodiment will be described, focusing on the differences from the biological signal measurement system 1 according to the first embodiment. In the first and second embodiments described above, the method of using the ABN-based trained model has been described. In the present embodiment, a method of using a model based on Deep U-Net will be described, which is a segmentation network. The overall configuration of the biological signal measurement system 1 and the hardware configuration of the information processing apparatus 50 according to the present embodiment are the same as those described in the first embodiment.

### Configuration of Functional Blocks and Operation of Information Processing Apparatus

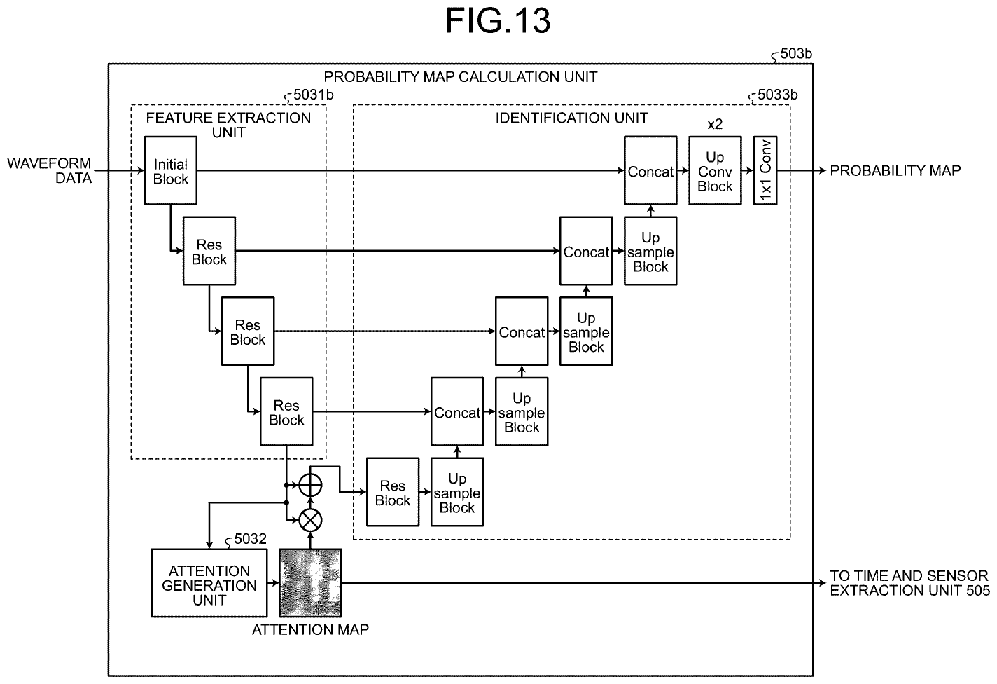

FIG. 13 is a diagram illustrating an example of the model configuration of the probability map calculation unit of the information processing apparatus according to the third embodiment. With reference to FIG. 13, the configuration of the functional blocks and the operation of the information processing apparatus 50 according to the present embodiment will be described.

The information processing apparatus 50 according to the present embodiment has a configuration in which the probability calculation unit 503 in the configuration of the functional blocks illustrated in FIG. 4 described above is replaced by the probability map calculation unit 503b illustrated in FIG. 13. Similar to the probability calculation units 503 and 503a described above, the probability map calculation unit 503b is a functional unit that calculates information on the probability at which characteristic waveform information of IED appears in the waveform data pre-processed by the pre-processing unit 502. Specifically, the probability map calculation unit 503b calculates the probability map by inference using a trained model based on a configuration on the basis of Deep U-Net (see, for example, Hirano, Ryoji, Takuto Emura, Otoichi Nakata, Toshiharu Nakashima, Miyako Asai, Kuriko Kagitani-Shimono, Haruhiko Kishima, and Masayuki Hirata. 2022. “Fully-Automated Spike Detection and Dipole Analysis of Epileptic MEG Using Deep Learning.” IEEE Transactions on Medical Imaging 41 (10): 2879-90.), which is a segmentation network as an example of a deep learning algorithm. The Deep U-Net consists of the first part serving as an encoder that extracts the feature map and the second part serving as a decoder that outputs the probability map based on the feature map output from the encoder. The deep learning algorithm used by the probability map calculation unit 503b is not limited to employing the Deep U-Net, and may also employ ordinary U-Net.

The probability map calculation unit 503b includes a feature extraction unit 5031b, an attention generation unit 5032, and an identification unit 5033b, as illustrated in FIG. 13. In FIG. 13, the feature extraction unit 5031b corresponds to the encoder of the Deep U-Net, and the identification unit 5033b corresponds to the decoder of the Deep U-Net. In other words, the configuration of the probability map calculation unit 503b illustrated in FIG. 13 has a configuration of adding the attention generation unit 5032 (attention branch) to the model configuration of the Deep U-Net.

The feature extraction unit 5031b is a functional unit that inputs the waveform data that has been pre-processed by the pre-processing unit 502 and generates a feature map by performing convolution processing over several stages (“Res Block” illustrated in FIG. 13) of the waveform data.

The attention generation unit 5032 is a functional unit that generates an attention map indicating an important region in IED recognition from the feature map generated by the feature extraction unit 5031b. Here, the feature map and the attention map have the same size. The attention map generated by the attention generation unit 5032 is output to the time and sensor extraction unit 505.

The identification unit 5033b is a functional unit that inputs information by multiplying the feature map output from the feature extraction unit 5031b by the attention map output from the attention generation unit 5032 to obtain the weighted feature map and furthermore adding the feature map itself to the weighted feature map, and identifies (calculates) a probability map indicating a distribution of the probability at which the characteristic waveform information of IED appears based on the input information. Specifically, the identification unit 5033b identifies (calculates) the probability map indicating the distribution of the probability at which the characteristic waveform information of IED appears by performing the reverse processing to the convolution processing on the input weighted feature map.

As described above, the model trained by the configuration based on Deep U-Net of the probability map calculation unit 503b is trained using the feature map weighted by multiplying the feature map related to the waveform data by the attention map indicating the important region in IED recognition. The labeled training data used for such training is the same as the labeled training data described in the first embodiment above. Accordingly, the weighted feature map is input to the identification unit 5033b, which is a decoder, to enable the training focusing on the important region in IED recognition indicated in the attention map and the output of the probability map of IED with high accuracy.

Although the information input to the identification unit 5033b is obtained by the feature map being weighted by multiplying the feature map by the attention map and furthermore adding the feature map itself thereto, this addition is not necessarily performed. However, the feature map with the region where the attention map responds strongly can be highlighted by adding the feature map itself. Thus, there is also the effect of not losing the feature map with the region where a value is 0 in the attention map.

### Fourth Embodiment

The biological signal measurement system 1 according to a fourth embodiment will be described, focusing on the differences from the biological signal measurement system 1 according to the third embodiment. In the present embodiment, regarding the waveform data in which characteristic waveform information of IED appears in addition to the labeled waveform data as the labeled training data used in the third embodiment, a method of using information (master information) on a time (sampling time) and a sensor recorded during the dipole estimation will be described. The overall configuration of the biological signal measurement system 1 and the hardware configuration of the information processing apparatus 50 according to the present embodiment are the same as those described in the first embodiment.

### Configuration of Functional Blocks and Operation of Information Processing Apparatus

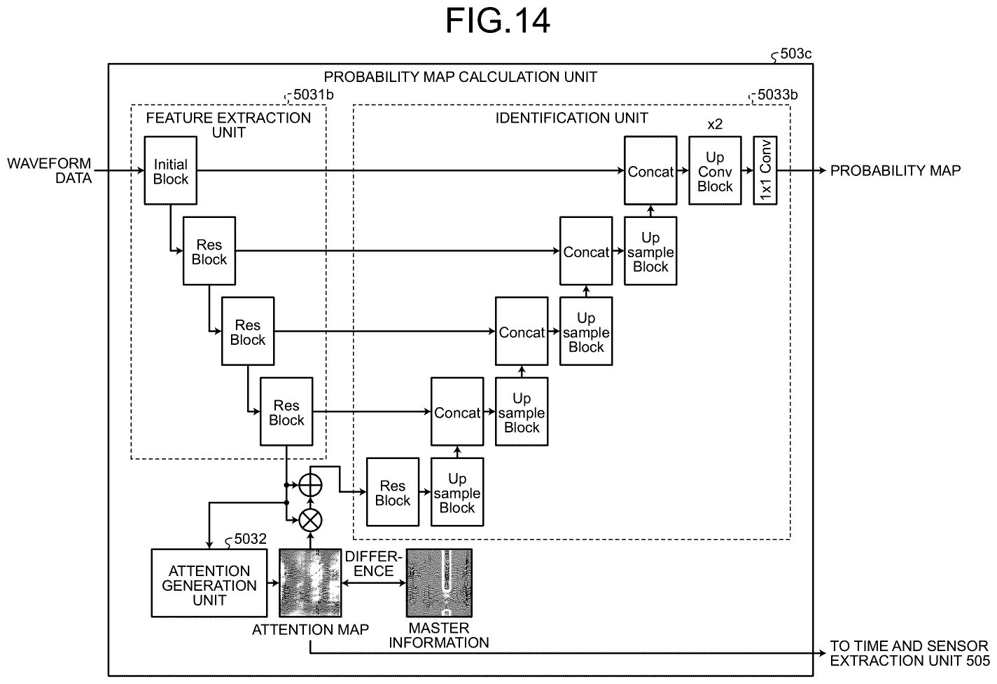

FIG. 14 is a diagram illustrating an example of the model configuration of the probability map calculation unit of the information processing apparatus according to the fourth embodiment. With reference to FIG. 14, the configuration of the functional blocks and the operation of the information processing apparatus 50 according to the present embodiment will be described.

The information processing apparatus 50 according to the present embodiment has a configuration in which the probability calculation unit 503 in the configuration of functional blocks illustrated in FIG. 4 above is replaced by the probability map calculation unit 503c illustrated in FIG. 14. The probability map calculation unit 503c includes a feature extraction unit 5031b, an attention generation unit 5032, and an identification unit 5033b, as illustrated in FIG. 14. The functions of the feature extraction unit 5031b and the identification unit 5033b are the same as those of the information processing apparatus 50 according to the third embodiment described above.

The attention generation unit 5032 is a functional unit that generates an attention map indicating an important region in IED recognition from the feature map generated by the feature extraction unit 5031b. Here, the feature map and the attention map have the same size. The attention map generated by the attention generation unit 5032 is output to the time and sensor extraction unit 505.

Here, regarding the waveform data in which characteristic waveform information of IED appears in addition to the labeled waveform data, as the labeled training data, the model trained by the configuration based on Deep U-Net of the probability map calculation unit 503c uses information on a time (sampling time) and a sensor recorded during the dipole estimation. The probability map calculation unit 503c then compares the attention map generated by the attention generation unit 5032 to the master information and performs training so that the difference between the attention map and the master information is reduced to generate a trained model.

In this way, since the training is performed with the master information that reflects the knowledge of experts included in the labeled training data, the experts' knowledge can be incorporated into the trained model. Accordingly, the more accurate analysis results can be extracted with respect to the time and sensors at which the characteristic waveform information of IED appears.

The comparison may also be performed by manually modifying the region to interest (important region in IED recognition) after the confirmation of the attention map output from the attention generation unit 5032 and comparing it with the modified attention map instead of using the master information prepared in advance.

In each of the above-described embodiments, provided that at least one of the functional units of the information processing apparatus 50 is implemented by execution of a computer program, the computer program is provided pre-embedded in ROM or the like. In each of the above-described embodiments, the computer program executed by the information processing apparatus 50 may be configured to be provided by recording the computer program as an installable file or an executable file in a computer-readable recording medium such as a compact disc read only memory (CD-ROM), a flexible disc (FD), a compact disc-recordable (CD-R), or a digital versatile disc (DVD). In each of the above-described embodiments, the computer program executed by the information processing apparatus 50 may be configured to be provided by storing the computer program in a computer connected to a network such as the Internet in a downloadable manner via the network. In each of the above-described embodiments, the computer program executed by the information processing apparatus 50 may be configured to be provided or distributed via a network such as the Internet. In each of the above-described embodiments, the computer program executed by the information processing apparatus 50 has a modular configuration including at least some of the above-described functional units. As the actual hardware, a CPU 101 reads the computer program from the storage device (such as the ROM 103 or the auxiliary storage device 104, for example) and executes the computer program, so that each of the functional units is loaded and generated in the main storage device (RAM 102).

Aspects of the present invention are as follows:
<1> A waveform generation identifying method including:
   acquiring waveform data of biological signals measured by a plurality of sensors;
   calculating probability information of appearance of IEDs (interictal epileptiform discharges) from a deep learning model trained using the waveform data with labels indicating whether characteristic waveform information appears or not; and
   first extracting a time and a sensor at which the characteristic waveform information appears in the waveform data, based on the probability information, wherein
   the calculating includes:
      second extracting a feature map indicating waveform data characteristics from the waveform data;
      generating an attention map indicating an important region in IED recognition, from the feature map; and
      identifying the probability information by inputting information obtained by multiplying the feature map by the attention map.
<2> The waveform generation identifying method according to <1>, wherein in the first extracting, the time and the sensor corresponding to the important region in IED recognition indicated in the attention map generated in the generating are extracted based on the probability information.
<3> The waveform generation identifying method according to <1> or <2>, wherein in the identifying, information obtained by multiplying the feature map by the attention map and furthermore adding the feature map is input to identify the probability information.
<4> The waveform generation identifying method according to <1> or <2>, further including determining whether the probability information calculated in the calculating is equal to or greater than a predetermined threshold value, wherein
   in the first extracting, the time and the sensor at which the characteristic waveform information appears in the waveform data are extracted based on the probability information determined as equal to or greater than the predetermined threshold value in the determining.
<5> The waveform generation identifying method according to <1> or <2>, wherein in the calculating, the deep learning model trained using master information to reduce a difference between the master information and the attention map generated in the generating is used, the master information being prepared in advance as information on the time and the sensor at which the characteristic waveform information appears.
<6> The waveform generation identifying method according to <1> or <2>, wherein in the calculating, the model trained using a manually modified version of the attention map generated in the generating is used.
<7> The waveform generation identifying method according to <1> or <2>, further including performing, on the waveform data acquired at the acquiring, pre-processing including processing of clipping out waveform data at least at a predetermined interval, wherein
   at the calculating, the probability information is calculated from the waveform data subjected to the pre-processing, using the model.
<8> The waveform generation identifying method according to <1> or <2>, wherein
   in the calculating, the model based on an attention branch network (ABN) is used,
   in the generating, processing is performed by a function of an attention branch of the ABN, and
   in the identifying, processing is performed by a function of a perception branch of the ABN.
<9> The waveform generation identifying method according to <1> or <2>, wherein
   in the calculating, the model based on Deep U-Net is used,
   in the second extracting, processing is performed by a function of an encoder of the Deep U-Net, and
   in the identifying, processing is performed by a function of a decoder of the Deep U-Net.
<10> A computer program causing a computer to execute:
   acquiring waveform data of biological signals measured by a plurality of sensors;
   calculating probability information on a probability at which characteristic waveform information of IED (interictal epileptiform discharge) appears, from the waveform data using a model of deep learning trained using, as training data, the acquired waveform data with a label indicating whether the characteristic waveform information appears; and
   first extracting a time and a sensor at which the characteristic waveform information appears in the waveform data, based on the probability information, wherein
the calculating includes:
   second extracting a feature map indicating waveform data characteristics from the waveform data;
   generating an attention map indicating an important region in IED recognition, from the feature map; and
   identifying the probability information by inputting information obtained by multiplying the feature map by the attention map.

According to an embodiment, the more accurate analysis results can be extracted with respect to the time and the sensor at which the characteristic waveform information of IED appears.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

1. A waveform generation identifying method including:
acquiring waveform data of biological signals measured by a plurality of sensors;
calculating probability information of appearance of IEDs (interictal epileptiform discharges) from a deep learning model trained using the waveform data with labels indicating whether characteristic waveform information appears or not; and
first extracting a time and a sensor at which the characteristic waveform information appears in the waveform data, based on the probability information, wherein
the calculating includes:
second extracting a feature map indicating waveform data characteristics from the waveform data;
generating an attention map indicating an important region in IED recognition, from the feature map; and
identifying the probability information by inputting information obtained by multiplying the feature map by the attention map.

2. The waveform generation identifying method according to claim 1, wherein in the first extracting, the time and the sensor corresponding to the important region in IED recognition indicated in the attention map generated in the generating are extracted based on the probability information.

3. The waveform generation identifying method according to claim 1, wherein in the identifying, information obtained by multiplying the feature map by the attention map and furthermore adding the feature map is input to identify the probability information.

4. The waveform generation identifying method according to claim 1, further including determining whether the probability information calculated in the calculating is equal to or greater than a predetermined threshold value, wherein
in the first extracting, the time and the sensor at which the characteristic waveform information appears in the waveform data are extracted based on the probability information determined as equal to or greater than the predetermined threshold value in the determining.

5. The waveform generation identifying method according to claim 1, wherein in the calculating, the deep learning model trained using master information to reduce a difference between the master information and the attention map generated in the generating is used, the master information being prepared in advance as information on the time and the sensor at which the characteristic waveform information appears.

6. The waveform generation identifying method according to claim 1, wherein in the calculating, the model trained using a manually modified version of the attention map generated in the generating is used.

7. The waveform generation identifying method according to claim 1, further including performing, on the waveform data acquired at the acquiring, pre-processing including processing of clipping out waveform data at least at a predetermined interval, wherein
at the calculating, the probability information is calculated from the waveform data subjected to the pre-processing, using the model.

8. The waveform generation identifying method according to claim 1, wherein
in the calculating, the model based on an attention branch network (ABN) is used,
in the generating, processing is performed by a function of an attention branch of the ABN, and
in the identifying, processing is performed by a function of a perception branch of the ABN.

9. The waveform generation identifying method according to claim 1, wherein
in the calculating, the model based on Deep U-Net is used,
in the second extracting, processing is performed by a function of an encoder of the Deep U-Net, and
in the identifying, processing is performed by a function of a decoder of the Deep U-Net.

10. A non-transitory computer readable medium storing a computer program for causing a computer to execute:
acquiring waveform data of biological signals measured by a plurality of sensors;
calculating probability information on a probability at which characteristic waveform information of IED (interictal epileptiform discharge) appears, from the waveform data using a model of deep learning trained using, as training data, the acquired waveform data with a label indicating whether the characteristic waveform information appears; and
first extracting a time and a sensor at which the characteristic waveform information appears in the waveform data, based on the probability information, wherein
the calculating includes:
second extracting a feature map indicating waveform data characteristics from the waveform data;
generating an attention map indicating an important region in IED recognition, from the feature map; and
identifying the probability information by inputting information obtained by multiplying the feature map by the attention map.
